(19) United States
(12) Patent Application Publication
(10) Pub. No.: US 2024/0428409 A1
(43) Pub. Date: Dec. 26, 2024

(54) ULTRASOUND DIAGNOSTIC APPARATUS AND PERFORMANCE MANAGEMENT METHOD

(71) Applicant: FUJIFILM Healthcare Corporation, Chiba (JP)

(72) Inventors: TOMOFUMI NISHIURA, Chiba (JP); Tsuyoshi Matsumoto, Tokyo (JP)

(73) Assignee: FUJIFILM Healthcare Corporation, Chiba (JP)

(21) Appl. No.: 18/747,390

(22) Filed: Jun. 18, 2024

(30) Foreign Application Priority Data
Jun. 26, 2023 (JP) 2023-104365

(51) Int. Cl. G06T 7/00

(52) U.S. Cl. CPC G06T 7/0012; G06T 2207/10132; G06T 2207/20081; G06T 2207/30168

(57) ABSTRACT

A recording unit records an analysis operation of an image analysis model and records an adoption/non-adoption of an analysis result. As a result, a log is generated. A calculation unit calculates an adoption rate as a score indicating performance of the image analysis model based on the log. A generation unit generates, in a case where the adoption rate is smaller than a threshold value, a reference image for notifying an examiner of such a situation.

### CROSS-REFERENCE TO RELATED APPLICATION

This application claims the priority benefits of Japanese application no. 2023-104365, filed on Jun. 26, 2023. The entirety of the above-mentioned patent application is hereby incorporated by reference herein and made a part of this specification.

### BACKGROUND

### 1. Technical Field

The present disclosure relates to an ultrasound diagnostic apparatus and a performance management method, and more particularly, to a technique of managing performance of an image analysis model.

### 2. Description of the Related Art

An ultrasound diagnostic apparatus is used in an ultrasound examination of a subject. The ultrasound diagnostic apparatus is a medical apparatus that generates and displays an ultrasound image based on a reception signal obtained by transmitting and receiving ultrasound waves.

In recent years, an ultrasound diagnostic apparatus having an image analysis model generated through machine learning has been increasingly prevalent. The image analysis model is configured with, for example, a convolutional neural network (CNN) that has been trained through machine learning. The image analysis model is, for example, a model that identifies a tissue cross section based on a tomographic image, or a model that performs measurement on a tissue image included in the tomographic image.

JP6423540B and JP2020-204970A disclose an image analysis model that identifies a tissue cross section based on a tomographic image. JP6423540B and JP2020-204970A do not disclose a technique of managing a temporal change in performance of the image analysis model. In the specification of the present application, a decrease in the performance of the image analysis model includes a decrease in the performance due to a decrease in quality of the ultrasound image.

### SUMMARY

The performance of the image analysis model depends on the quality of the machine learning and also changes depending on the quality of the ultrasound image to be input. In any case, in a case where the decrease in the performance of the image analysis model is recognized, it is desired to notify an examiner (a user such as a doctor or an examination technician) of such a situation and prompt the examiner to take appropriate measures.

An object of the present disclosure is to allow an examiner to recognize such a situation in a case where the performance of the image analysis model is decreased. Alternatively, an object of the present disclosure is to prompt the examiner to take appropriate measures in a case where the performance of the image analysis model is decreased.

According to the present disclosure, there is provided an ultrasound diagnostic apparatus comprising: an analysis unit that includes an image analysis model generated through machine learning, sequentially analyzes a plurality of ultrasound images, and sequentially outputs a plurality of analysis results; a recording unit that records a plurality of analysis operations of the image analysis model and records adoptions/non-adoptions of the plurality of analysis results to generate a log including a first record column consisting of a plurality of analysis operation records and a second record column consisting of a plurality of adoption/non-adoption records; a calculation unit that calculates a score indicating performance of the image analysis model based on the first record column and the second record column; and a generation unit that generates reference information to be provided to an examiner in accordance with the score, in which the reference information includes at least one of information representing a decrease in the performance of the image analysis model or information for prompting a determination of an adoption/non-adoption of a current analysis result.

According to the present disclosure, there is provided a performance management method comprising: a step of sequentially analyzing a plurality of ultrasound images by using an image analysis model generated through machine learning, thereby sequentially generating a plurality of analysis results; a step of recording a plurality of analysis operations of the image analysis model and recording adoptions/non-adoptions of the plurality of analysis results to generate a log including a first record column consisting of a plurality of analysis operation records and a second record column consisting of a plurality of adoption/non-adoption records; a step of calculating a score indicating performance of the image analysis model based on the first record column and the second record column; and a step of generating reference information to be provided to an examiner in accordance with the score, in which the reference information includes at least one of information representing a decrease in the performance of the image analysis model or information for prompting a determination of an adoption/non-adoption of a current analysis result.

According to the present disclosure, it is possible to allow the examiner to recognize such a situation in a case where the performance of the image analysis model is decreased. Alternatively, according to the present disclosure, it is possible to prompt the examiner to take appropriate measures in a case where the performance of the image analysis model is decreased.

### BRIEF DESCRIPTION OF THE DRAWINGS

FIG. 1 is a block diagram showing an ultrasound diagnostic apparatus according to an embodiment.

FIG. 2 is a block diagram showing a configuration example of an image analysis unit.

FIG. 3 is a diagram showing an example of a log.

FIG. 4 is a diagram showing an adoption rate graph.

FIG. 5 is a diagram showing a display example.

FIG. 6 is a flowchart showing a performance management method according to the embodiment.

FIG. 7 is a diagram showing a first example of a popup window.

FIG. 8 is a diagram showing a second example of the popup window.

FIG. 9 is a diagram showing another popup window.

FIG. 10 is a diagram showing an image evaluation method.

FIG. 11 is a diagram showing another example of the log.

### DESCRIPTION OF THE EMBODIMENTS

Hereinafter, an embodiment will be described with reference to the drawings.

### (1) Outline of Embodiment

An ultrasound diagnostic apparatus according to the embodiment includes an analysis unit, a recording unit, a calculation unit, and a generation unit. The analysis unit includes an image analysis model generated through machine learning, sequentially analyzes a plurality of ultrasound images, and sequentially outputs a plurality of analysis results. The recording unit records a plurality of analysis operations of the image analysis model and records adoptions/non-adoptions of the plurality of analysis results to generate a log including a first record column consisting of a plurality of analysis operation records and a second record column consisting of a plurality of adoption/non-adoption records. The calculation unit calculates a score indicating performance of the image analysis model based on the first record column and the second record column. The generation unit generates reference information to be provided to an examiner in accordance with the score. The reference information includes at least one of information representing a decrease in the performance of the image analysis model or information for prompting a determination of an adoption/non-adoption of a current analysis result.

With the above-described configuration, the reference information is generated in a case where the performance of the image analysis model is changed, and the reference information is provided to the examiner. The examiner recognizes the change in the performance of the image analysis model through the visual recognition of the reference information. Consequently, it is possible to prompt the examiner to take appropriate measures. For example, it is possible to prompt the examiner to correct or reject the analysis result, to perform a probe operation to enhance ultrasound image quality, or the like.

In a case where the reference information is always provided to the examiner, the reference information may hinder the ultrasound examination (for example, being visually distracting). In the embodiment, the generation unit generates the reference information in a case where the score falls below a set threshold value. That is, the reference information is displayed in a case where the score falls below the threshold value.

The image analysis model is a model that identifies a type of a tissue cross section, a model that performs measurement on a tissue image, a model that specifies a lesion part, a model that analyzes the lesion part, or the like. Each analysis operation record indicates a fact that the analysis operation is executed. Each adoption/non-adoption record indicates an adoption or a non-adoption of the analysis result. Assuming that each individual analysis operation is recorded, an adoption record is also a non-adoption record, and conversely, a non-adoption record is also an adoption record. Therefore, only the adoption may be recorded, only the non-adoption may be recorded, or both the adoption and the non-adoption may be recorded. The score is information indicating the magnitude of the performance of the image analysis model, in other words, information indicating the magnitude of the reliability of the image analysis result. In the embodiment, the score is calculated based on the log as described above, that is, based on objectively specified past performance records.

In the embodiment, the calculation unit calculates an adoption rate as the score based on the number of analysis operations within a certain period specified from the first record column and the number of adoptions within a certain period specified from the second record column. For example, the adoption rate is calculated by dividing the number of adoptions by the number of analysis operations. The adoption rate can also be calculated by dividing a numerical value (the number of adoptions) obtained by subtracting the number of non-adoptions from the number of analysis operations by the number of analysis operations. In a case where the number of non-adoptions is smaller than the number of adoptions, it is more rational to record non-adoption. The adoption rate can also be referred to as an effective operation rate, an effective use frequency, and the like. The adoption rate is information representing a non-adoption rate in a sense.

In the embodiment, each adoption/non-adoption record is a non-adoption record representing correction or rejection of the analysis result by the examiner. The evaluation of the analysis result is usually performed by the examiner, and in a case where the analysis result is deemed invalid, the analysis result is corrected by the examiner or the analysis result is rejected by the examiner. By recording such a determination or action of the examiner, it is possible to calculate the score.

In the embodiment, each analysis operation record includes information representing a time at which the analysis operation is executed. Each adoption/non-adoption record includes information representing a time at which the adoption/non-adoption of the analysis result is input. With this configuration, it is possible to accurately specify the number of analysis operations and the number of adoptions, which are calculation targets.

The ultrasound diagnostic apparatus according to the embodiment further includes an evaluation unit that determines whether or not each ultrasound image input to the analysis unit satisfies a predetermined image quality condition. The number of analysis operations described above is the number of a plurality of analysis operations corresponding to a plurality of ultrasound images that satisfy the image quality condition. The number of adoptions described above is the number of one or a plurality of analysis results adopted by the examiner among a plurality of analysis results corresponding to the plurality of ultrasound images that satisfy the image quality condition.

With the above-described configuration, for example, an analysis error resulting from a significant decrease in the quality of the ultrasound image can be excluded from the aggregation target. Therefore, the score can be accurately calculated.

In the embodiment, the recording unit records an analysis operation corresponding to an ultrasound image that satisfies the image quality condition and does not record an analysis operation corresponding to an ultrasound image that does not satisfy the image quality condition. In addition, the recording unit records an adoption/non-adoption of an analysis result corresponding to the ultrasound image that satisfies the image quality condition and does not record an adoption/non-adoption of an analysis result corresponding to the ultrasound image that does not satisfy the image quality condition.

With the above-described configuration, the number of records included in the log can be reduced, or unnecessary records for the log can be avoided. All the analysis operations and all the adoptions/non-adoptions may be recorded while recording whether or not the image quality condition is satisfied.

In the embodiment, the first record column includes a plurality of subject identifiers associated with the plurality of analysis operation records. The second record column includes a plurality of subject identifiers associated with the plurality of adoption/non-adoption records. The calculation unit calculates a score for each subject based on the first record column and the second record column. The content of the ultrasound image changes depending on the physique or the tissue properties of the subject. With the above-described configuration, it is possible to appropriately determine whether or not to generate and display the reference information for each subject.

A performance management method according to the embodiment includes an analysis step, a recording step, a calculation step, and a generation step. In the analysis step, a plurality of ultrasound images are sequentially analyzed by using the image analysis model generated through machine learning, thereby sequentially generating a plurality of analysis results. In the recording step, a plurality of analysis operations of the image analysis model are recorded, and adoptions/non-adoptions of the plurality of analysis results are recorded. Consequently, a log including the first record column consisting of the plurality of analysis operation records and the second record column consisting of the plurality of adoption/non-adoption records is generated. In the calculation step, a score indicating the performance of the image analysis model is calculated based on the first record column and the second record column. In the generation step, the reference information to be provided to the examiner is generated in accordance with the score. The reference information includes at least one of information representing a decrease in the performance of the image analysis model or information for prompting a determination of an adoption/non-adoption of a current analysis result.

A program for executing the above-described performance management method is installed in the ultrasound diagnostic apparatus serving as an information processing apparatus via a network or a portable storage medium. The ultrasound diagnostic apparatus includes a storage medium that non-transitorily stores the installed program.

### (2) Details of Embodiment

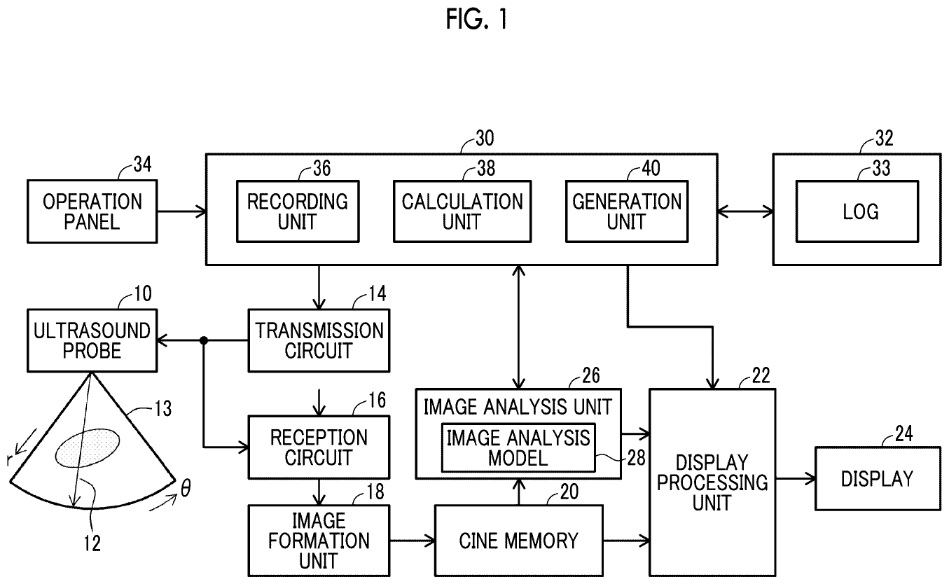

FIG. 1 shows the ultrasound diagnostic apparatus according to the embodiment. This ultrasound diagnostic apparatus is a medical apparatus that is installed in a medical institution, such as a hospital, and that is used in the ultrasound examination of the subject.

An ultrasound probe 10 is a device that transmits ultrasound waves into a living body and that receives reflected waves from the living body. The ultrasound probe 10 includes a transducer array consisting of a plurality of transducers. The transducer array forms an ultrasound beam 12. A beam scanning plane 13 is formed through electronic scanning of the ultrasound beam 12. The beam scanning plane 13 is repeatedly formed by repeating the electronic scanning with the ultrasound beam 12 in accordance with the transmission frame rate. In FIG. 1, an r direction is a depth direction, and a 0 direction is an electronic scanning direction.

As an electronic scanning method of the ultrasound beam 12, an electronic linear scanning method, an electronic sector scanning method, and the like are known. A two-dimensional transducer array may be provided as the transducer array. Volume data can be acquired from a three-dimensional space in the living body by performing two-dimensional scanning with the ultrasound beam using the two-dimensional transducer array.

A transmission circuit 14 is an electronic circuit that functions as a transmission beam former, and outputs a plurality of transmission signals to the transducer array in parallel during transmission. As a result, a transmission beam is formed by the action of the transducer array.

A reception circuit 16 is an electronic circuit that functions as a reception beam former, and applies phase addition to a plurality of reception signals output in parallel from the transducer array during reception, thereby generating reception beam data. With the repetition of the electronic scanning, a reception frame data column is output from the reception circuit 16. Each piece of the reception frame data is composed of a plurality of pieces of reception beam data arranged in the electronic scanning direction. Each piece of the reception beam data is composed of a plurality of pieces of echo data arranged in the depth direction.

The reception frame data column is sent to an image formation unit 18 through a data processing unit (not shown). The data processing unit is a module that applies a plurality of kinds of processing to each individual piece of the reception beam data. The plurality of kinds of processing include logarithmic transformation, filtering, and the like.

The image formation unit 18 includes a digital scan converter (DSC). The DSC has a coordinate transformation function, a pixel interpolation function, and the like. A display frame data column is generated from the reception frame data column by the DSC. The display frame data column corresponds to a tomographic image as a moving image. Each individual piece of the display frame data constituting the display frame data column corresponds to a tomographic image as a still image.

In the shown configuration example, the display frame data column is stored in a cine memory 20, and then the display frame data column read out from the cine memory 20 is sent to a display 24 via a display processing unit 22. The tomographic image as a moving image is displayed on the display 24, or the tomographic image as a still image is displayed on the display 24. The display processing unit 22 has an image combining function, a color processing function, and the like.

The above-described cine memory 20 has a ring buffer structure. The cine memory 20 is configured with, for example, a semiconductor memory. The image formation unit 18 and the display processing unit 22 are each configured with, for example, a processor. The display 24 is configured with a liquid crystal display, an organic EL device, or the like. In the image formation unit 18, an ultrasound image other than the tomographic image may be formed. For example, a blood flow image, an elasticity image, or the like may be formed.

An image analysis unit 26 includes an image analysis model 28. The image analysis model 28 is a model that has been trained through machine learning, and is configured with, for example, a CNN. The image analysis model 28 according to the embodiment is a model that identifies a tissue cross section based on the tomographic image. As the image analysis model 28, a model that executes measurement on the tissue based on the tomographic image, a model that detects a lesion part included in the tomographic image, or the like may be used.

In the embodiment, the display frame data column is transferred from the cine memory 20 to the image analysis unit 26. The image analysis model 28 executes image analysis on each piece of the display frame data constituting the display frame data column for each piece of the display frame data (that is, for each tomographic image) and outputs an analysis result for each piece of the display frame data. An analysis result column corresponding to the display frame data column is output from the image analysis unit 26 to the display processing unit 22 and an information processing unit 30. The analysis result column is sent to the display 24 via the display processing unit 22, and the analysis result column is displayed on the display 24. Alternatively, in the information processing unit 30, predetermined processing is executed based on the analysis result column. The image analysis unit 26 is configured with, for example, a processor. The image analysis unit 26 may analyze the reception frame data.

The information processing unit 30 is configured with, for example, a CPU that executes a program. The information processing unit 30 functions as a controller that controls an operation of each element constituting the ultrasound diagnostic apparatus. In the drawing, a plurality of functions exerted by the information processing unit 30 are represented by a plurality of blocks. Specifically, the information processing unit 30 functions as a recording unit 36, a calculation unit 38, and a generation unit 40. These will be described in detail below.

An operation panel 34 is connected to the information processing unit 30. The operation panel 34 is an input device including a track ball, a plurality of switches, a plurality of knobs, and the like. The examiner uses the operation panel 34 to input the adoption/non-adoption of the analysis result output by the image analysis model, or to correct the analysis result.

A storage unit 32 is connected to the information processing unit 30. The storage unit 32 is configured with a semiconductor memory, a hard disk, or the like. In the embodiment, a log 33 for managing a temporal change in the performance of the image analysis model 28 is constructed on the storage unit 32.

The recording unit 36 records the fact of the analysis operation of the image analysis model 28 on the log 33 for each analysis operation. In addition, the recording unit 36 records the fact of the adoption/non-adoption of the analysis result on the log 33 for each analysis operation of the image analysis model 28. As will be described below, the log 33 includes a first record list and a second record list. The first record list is composed of a plurality of analysis operation records arranged in time series order. The second record list is composed of a plurality of adoption/non-adoption records arranged in time series order. Each individual adoption/non-adoption record is actually a non-adoption record in the embodiment.

The calculation unit 38 calculates the adoption rate as the score indicating the performance of the image analysis model. The calculation unit 38 specifies the number of analysis operations within a certain period in the past from the current point in time based on the first record list and specifies the number of non-adoptions within the same certain period based on the second record list. Then, the calculation unit 38 calculates the adoption rate from the number of analysis operations and the number of non-adoptions. The adoption rate is an evaluation value in which the performance record of the image analysis model is reflected.

The generation unit 40 generates a reference image as the reference information in a case where the adoption rate as the score falls below a set threshold value. The reference image is sent to the display 24 via the display processing unit 22. As will be described below, the reference image is displayed on the display 24 as a popup window. The reference image includes first information for notifying the examiner of a decrease in the adoption rate and second information for prompting a determination of the adoption/non-adoption of the analysis result. Each information is text, a figure, a mark, or the like.

In a case where the adoption rate is larger than the threshold value, the reference image is not displayed. The correction or rejection of the analysis result is always accepted. The information processing unit 30 may function as the image analysis unit 26 and the display processing unit.

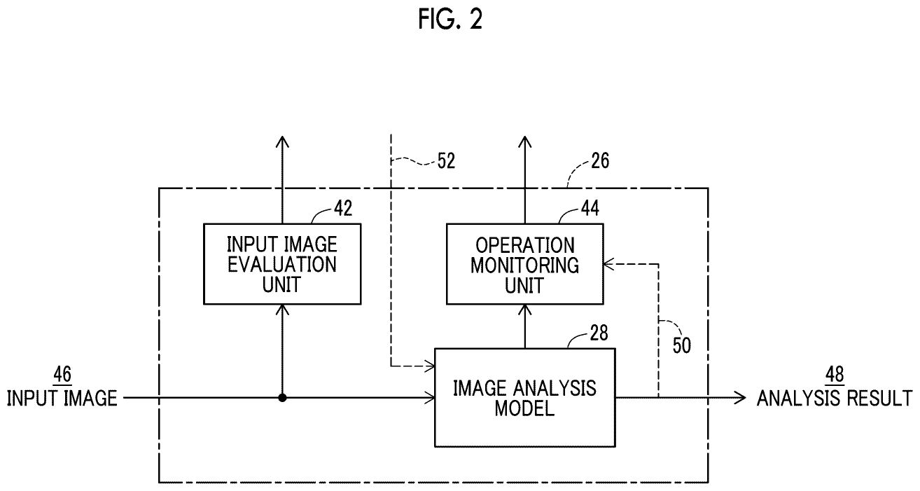

FIG. 2 shows a configuration example of the image analysis unit 26. The image analysis unit 26 includes the image analysis model 28 generated through machine learning. In a case where the tomographic image is provided to the image analysis model 28 as an input image 46, an analysis result 48 is output from the image analysis model 28. The analysis result 48 is, for example, a cross section identification result.

The image analysis unit 26 includes an input image evaluation unit 42 and an operation monitoring unit 44, in addition to the image analysis model 28. The operation monitoring unit 44 is a module that monitors the analysis operation of the image analysis model and that reports a fact that the analysis operation is executed to the information processing unit each time the analysis operation is executed. As indicated by a reference numeral 50, the analysis result may be transferred to the information processing unit by the operation monitoring unit 44. As indicated by a reference numeral 52, retraining of the image analysis model may be executed as necessary. For example, in a case where the performance of the image analysis model is decreased, it may be determined to retrain the image analysis model.

The input image evaluation unit 42 is a module that evaluates the quality of each input image 46. Specifically, the input image evaluation unit 42 determines whether or not each input image 46 satisfies the image quality condition. The determination result is sent to the information processing unit. The analysis operation is recorded on the log only in a case where the input image 46 satisfies the image quality condition, and the adoption/non-adoption is recorded on the log. In a case where all or a part of a plurality of conditions set in advance are satisfied, or in a case where at least one of the conditions set in advance is satisfied, it may be determined that the input image 46 satisfies the image quality condition.

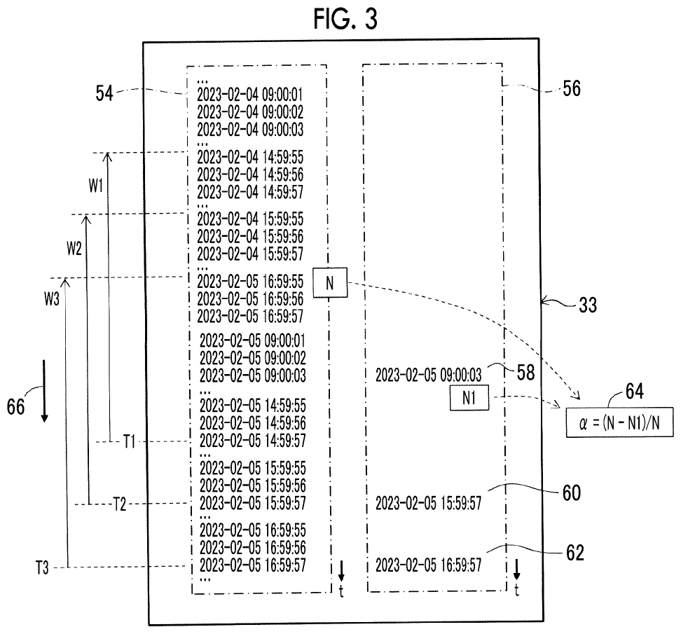

FIG. 3 shows an example of the log. The log 33 is composed of a first record list 54 and a second record list 56. The log 33 is updated at any time by the recording unit described above.

The first record list 54 is composed of a plurality of analysis operation records arranged in time series order. In the embodiment, one analysis operation record is created for one analysis operation of the image analysis model. Each individual analysis operation record is actually information for specifying a time at which the analysis operation is executed, and includes information indicating the year, month, and day and information indicating the hour, minute, and second. The plurality of analysis operations may be recorded at constant sampling intervals.

The second record list 56 is composed of a plurality of non-adoption records 58, 60, and 62 arranged in time series order. The individual non-adoption records 58, 60, and 62 are each information for specifying a time at which the correction or rejection of the analysis result is input. Each of the individual non-adoption records 58, 60, and 62 includes information indicating the year, month, and day and information indicating the hour, minute, and second. A plurality of non-adoptions may be recorded at constant sampling intervals.

The calculation unit described above calculates the non-adoption rate based on the log 33. Specifically, the calculation unit sets individual time ranges W1, W2, and W3 covering a certain period in the past from the current time at individual calculation times T1, T2, and T3. Subsequently, the calculation unit aggregates the number of analysis operation records included in each of the time ranges W1, W2, and W3 for each of the time ranges W1, W2, and W3 based on the first record list 54. An aggregation result thereof is the number of analysis operations, which is denoted by N.

Meanwhile, the calculation unit aggregates the number of non-adoption records included in each of the time ranges W1, W2, and W3 for each of the time ranges W1, W2, and W3 based on the second record list 56. An aggregation result thereof is the number of non-adoptions, which is denoted by N1. The calculation unit calculates an adoption rate α by calculating (N−N1)/N at each of the individual calculation times T1, T2, and T3 (refer to a reference numeral 64). An adoption rate graph is generated from a plurality of adoption rates calculated at the plurality of calculation times T1, T2, and T3.

In the above-described calculation expression, (N−N1) corresponds to the number of adoptions. Assuming that each analysis operation is recorded, the non-adoption record is equal to the adoption record. In general, since the number of adoptions is larger than the number of non-adoptions, it is more rational to record the non-adoption. The above-described time range corresponds to an aggregation period. The time range and a period for calculating the non-adoption rate can be freely set. For example, the time range may be set to 24 hours, or the period may be set to 1 hour. A period for recording the analysis operation may be set to 1 second.

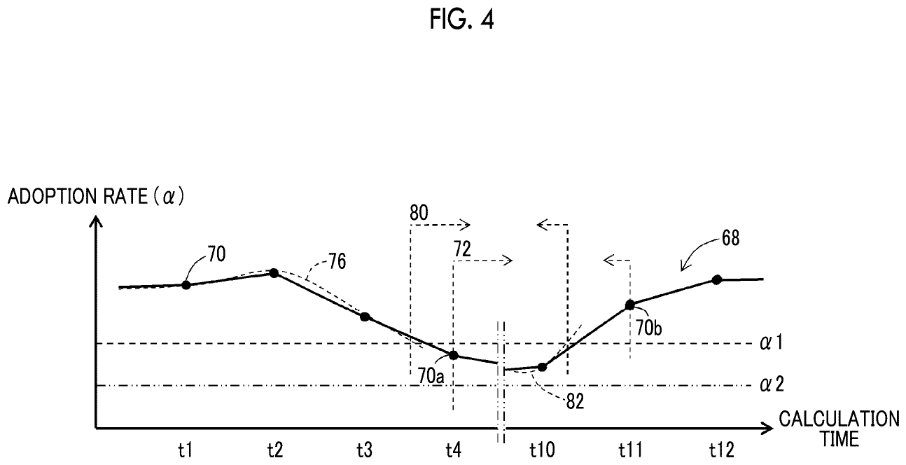

FIG. 4 shows an example of the adoption rate graph. A horizontal axis is a time axis, and a vertical axis indicates the adoption rate. A plurality of adoption rates are calculated at a plurality of calculation times. An adoption rate graph 68 is generated by plotting the plurality of adoption rates as a plurality of points 70.

A threshold value α1 is set for the adoption rate graph 68. In a case where the calculated adoption rate (refer to a reference numeral 70a) falls below the threshold value α1, the reference image (popup window) for notifying the examiner of the adoption rate is displayed. For example, in the example shown in FIG. 4, as indicated by a reference numeral 72, the reference image is displayed after t4 with the display of the analysis result. In t4 and later, the reference image may be always displayed. In a case where the calculated adoption rate (refer to a reference numeral 70b) exceeds the threshold value α1, the reference image is no longer displayed.

An approximation curve 76 may be generated based on the plurality of points 70 constituting the adoption rate graph 68, and the reference image may be displayed after a point in time at which the approximation curve 76 falls below the threshold value α1 (refer to a reference numeral 80). In that case, the display of the reference image may be ended at a point in time at which an approximate curve 82 exceeds the threshold value α1. A plurality of threshold values α1 and α2 may be set, and the content or the aspect of the reference image may be switched according to a section to which the calculated adoption rate belongs.

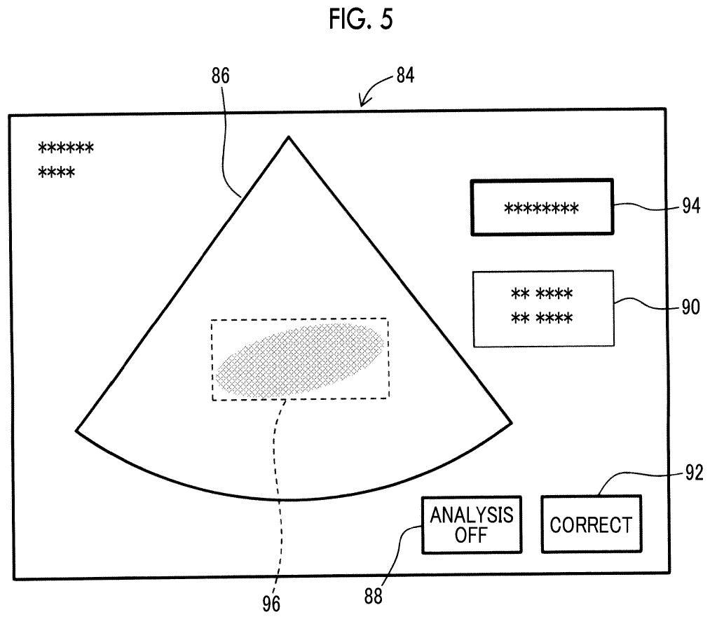

FIG. 5 shows a display example. An image 84 displayed on the display includes a tomographic image 86. The tomographic image 86 is, specifically, a frozen tomographic image. For example, the ultrasound diagnostic apparatus automatically enters a frozen state at a point in time at which a specific cross section is recognized. Here, the detection of the lesion part is also executed. The lesion part is surrounded by a box 96.

A window 90 includes text information indicating the recognized cross section. A button 88 is operated at the start or end of the image analysis. A button 92 is operated in a case of correcting a cross section recognition result. This is a button for rejecting the cross section recognition result. In a case of correcting the cross section recognition result, an operation panel or a touch screen panel is used.

In the embodiment, a reference image 94 is displayed together with the cross section recognition result in a state in which the adoption rate is smaller than the threshold value. The reference image 94 includes first information representing that the adoption rate is decreased and second information for prompting a determination of the adoption/non-adoption of the analysis result. By displaying the reference image 94, the examiner can recognize that the adoption rate is decreased (or that there is a concern that the reliability of the analysis result is decreased), and the examiner can be prompted to take necessary measures.

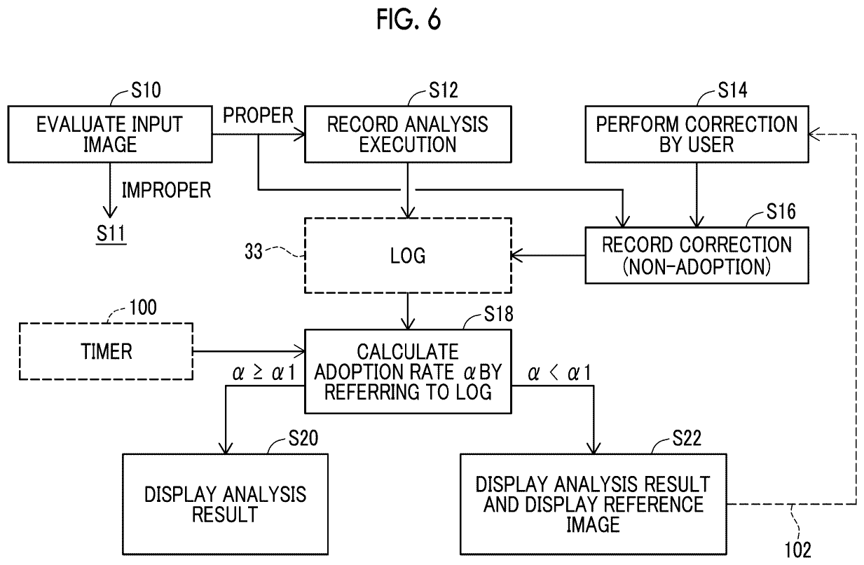

FIG. 6 shows a flowchart of a performance management method according to the embodiment. This performance management method is executed by the ultrasound diagnostic apparatus shown in FIG. 1.

In 510, an input image is evaluated. In a case where the input image satisfies the predetermined image quality condition, it is determined that the input image is a proper image, and the execution of S12, which will be described below, is allowed, and the execution of S16, which will be described below, is allowed. On the other hand, in a case where the input image does not satisfy the predetermined image quality condition, in S11, the recording of the analysis execution is restricted, and the recording of the correction (non-adoption) is restricted. In that case, the image analysis itself may be restricted.

In S12, the fact of the analysis execution is recorded in the log 33. Specifically, the analysis operation record is added to the log 33. In S14, the analysis result is corrected by the examiner. With such actions assumed, in S16, the fact of the correction (non-adoption) is recorded in the log 33. Specifically, the non-adoption record is added to the log 33.

In S18, the log is referred to, and the adoption rate α is calculated based on the log. In the embodiment, S18 is executed every certain time based on time information generated by a timer 100. In a case where the adoption rate α is equal to or more than the threshold value α1, the analysis result is displayed in S20. In that case, the reference image is not displayed. On the other hand, in a case where the adoption rate α is smaller than the threshold value α1, the analysis result is displayed in S22, and the reference image is displayed at the same time. As indicated by a reference numeral 102, the analysis result is corrected in S14 as necessary by the examiner who views the reference image.

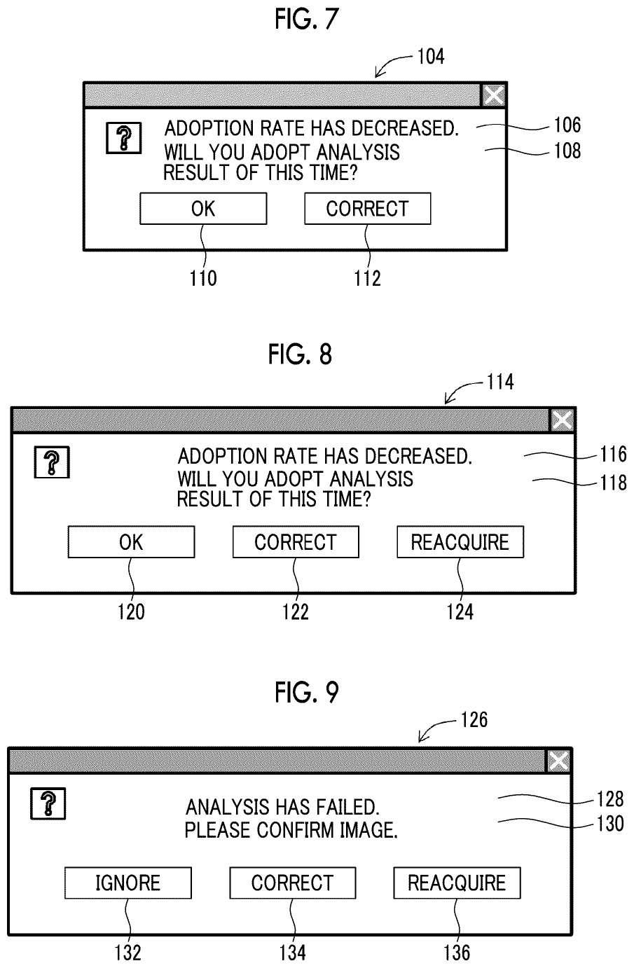

FIGS. 7 and 8 show specific examples of the reference image. In a first example shown in FIG. 7, the reference image is specifically a popup window 104 that is displayed in a pop-up manner. The popup window 104 is displayed together with the analysis result. The popup window 104 includes text information 106 indicating a decrease in the adoption rate and text information 108 for prompting a determination of the adoption/non-adoption of the analysis result of this time. In a case of approving the analysis result of this time, a button 110 is operated, and in a case of correcting, that is, rejecting the analysis result of this time, a button 112 is operated.

In a second example shown in FIG. 8, a popup window 114 includes text information 116 indicating a decrease in the adoption rate and text information 118 for prompting a determination of the adoption/non-adoption of the analysis result of this time. In a case of approving the analysis result of this time, a button 120 is operated, and in a case of correcting, that is, rejecting the analysis result of this time, a button 122 is operated. In a case of releasing the frozen state and reacquiring the image (in a case of requesting reanalysis), a button 124 is operated.

In a case where the input image evaluation unit (refer to FIG. 2) determines that the input image is not proper, a popup window 126 shown in FIG. 9 may be displayed. This popup window 126 is displayed together with a window showing the frozen tomographic image and the analysis result. The popup window 126 includes text information 128 indicating analysis failure and text information 130 for requesting image confirmation. In a case of ignoring such an alert, a button 132 is operated. In a case of correcting, that is, rejecting the analysis result of this time, a button 134 is operated. In a case of releasing the frozen state and reacquiring the image (in a case of requesting reanalysis), a button 136 is operated.

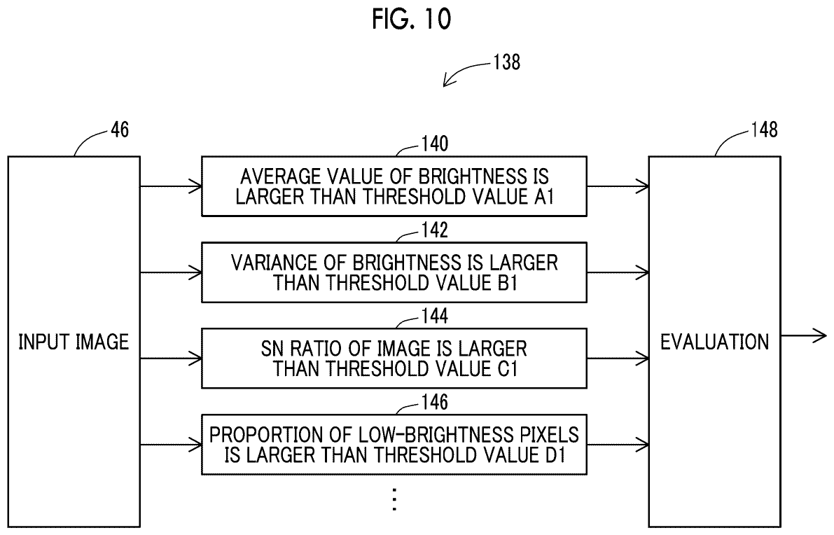

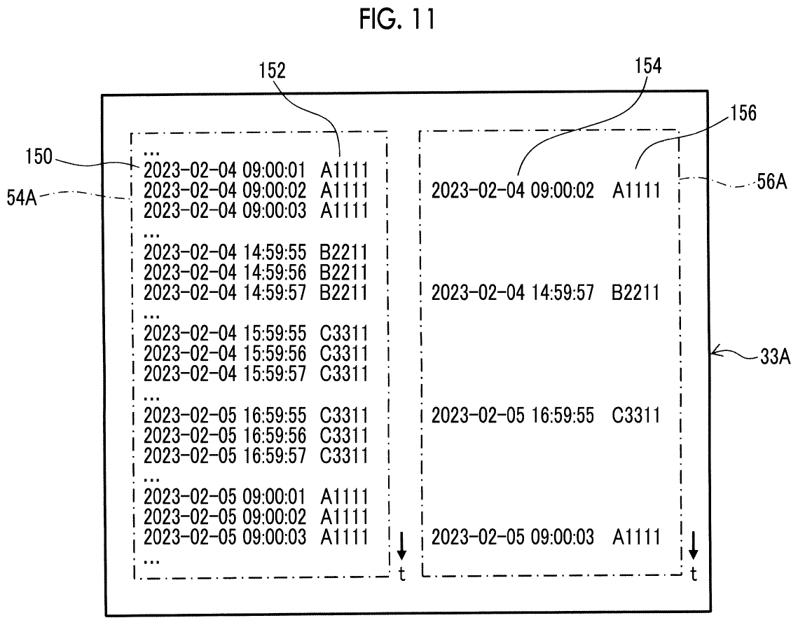

FIG. 10 shows an evaluation method of the input image. A reference numeral 138 indicates the image quality condition. The image quality condition includes a plurality of conditions 140, 142, 144, and 146 in the shown example. In the shown example, for example, in a case where all the conditions 140, 142, 144, and 146 are satisfied, it is determined that the input image satisfies the image quality condition (refer to a reference numeral 148). In a case where any one of the plurality of conditions 140, 142, 144, and 146 is not satisfied, it is determined that the input image does not satisfy the image quality condition (refer to a reference numeral 148).

In the shown example, the condition 140 requires that the average value of the brightness included in the input image is larger than a threshold value A1. The condition 140 excludes the analysis operation for a dark image from a recording target. The condition 142 requires that the variance of the brightness included in the input image is larger than a threshold value B1. The condition 142 excludes the analysis operation for an unclear image from the recording target. The condition 144 requires that the SN ratio of the input image is larger than a threshold value Cl. The condition 144 excludes the analysis operation for an image having a large amount of noise from the recording target. The condition 146 requires that the proportion of low-brightness pixels within all the pixels constituting the input image is larger than a threshold value D1. The condition 146 excludes the analysis operation for an image including a shadow from the recording target. For example, the shadow is generated in the tomographic image in a case where a part of a transmission/reception wave surface of the ultrasound probe is separated from a body surface. Each of the above conditions is an example, and the image quality condition can be freely set.

FIG. 11 shows another example of the log. A log 33A is composed of a first record list 54A and a second record list 56A. The first record list 54A is composed of a plurality of analysis operation records 150 arranged in time series order and a plurality of subject identifiers 152 associated with the plurality of analysis operation records 150. Each individual subject identifier 152 is, for example, a subject code.

The second record list 56A is composed of a plurality of non-adoption records 154 and a plurality of subject identifiers 156 associated with the plurality of non-adoption records. Since the first record list 54A includes the plurality of subject identifiers 152, the plurality of subject identifiers 156 may be deleted from the second record list 56A.

By constructing the log 33A, it is possible to calculate the adoption rate as the score for each subject by using the method shown in FIG. 3. The content of the input image changes according to the physique and the tissue properties of the subject. Therefore, in a case where the adoption rate is calculated for each subject, the reference image can be displayed at a more appropriate timing.

According to the embodiment, in a case where the performance of the image analysis model is decreased, it is possible to allow the examiner to recognize such a situation. In addition, in a case where the performance of the image analysis model is decreased, it is possible to prompt the examiner to take appropriate measures.

What is claimed is:

1. An ultrasound diagnostic apparatus comprising:
an analysis unit that includes an image analysis model generated through machine learning, sequentially analyzes a plurality of ultrasound images, and sequentially outputs a plurality of analysis results;
a recording unit that records a plurality of analysis operations of the image analysis model and records adoptions/non-adoptions of the plurality of analysis results to generate a log including a first record column consisting of a plurality of analysis operation records and a second record column consisting of a plurality of adoption/non-adoption records;
a calculation unit that calculates a score indicating performance of the image analysis model based on the first record column and the second record column; and
a generation unit that generates reference information to be provided to an examiner in accordance with the score,
wherein the reference information includes at least one of information representing a decrease in the performance of the image analysis model or information for prompting a determination of an adoption/non-adoption of a current analysis result.

2. The ultrasound diagnostic apparatus according to claim 1,
wherein the generation unit generates the reference information in a case where the score falls below a set threshold value.

3. The ultrasound diagnostic apparatus according to claim 1,
wherein the calculation unit calculates an adoption rate as the score based on the number of analysis operations within a certain period specified from the first record column and the number of adoptions within the certain period specified from the second record column.

4. The ultrasound diagnostic apparatus according to claim 1,
wherein each adoption/non-adoption record is a non-adoption record representing correction or rejection of the analysis result by the examiner.

5. The ultrasound diagnostic apparatus according to claim 1,
wherein each analysis operation record includes information representing a time at which the analysis operation is executed, and
each adoption/non-adoption record includes information representing a time at which the adoption/non-adoption of the analysis result is input.

6. The ultrasound diagnostic apparatus according to claim 3, further comprising:
an evaluation unit that determines whether or not each ultrasound image input to the analysis unit satisfies a predetermined image quality condition,
wherein the number of analysis operations is the number of a plurality of analysis operations corresponding to a plurality of ultrasound images that satisfy the image quality condition, and
the number of adoptions is the number of one or a plurality of analysis results adopted by the examiner among a plurality of analysis results corresponding to the plurality of ultrasound images that satisfy the image quality condition.

7. The ultrasound diagnostic apparatus according to claim 6,
wherein the recording unit
records an analysis operation corresponding to an ultrasound image that satisfies the image quality condition and does not record an analysis operation corresponding to an ultrasound image that does not satisfy the image quality condition, and
records an adoption/non-adoption of an analysis result corresponding to the ultrasound image that satisfies the image quality condition and does not record an adoption/non-adoption of an analysis result corresponding to the ultrasound image that does not satisfy the image quality condition.

8. The ultrasound diagnostic apparatus according to claim 1,
wherein the first record column includes a plurality of subject identifiers associated with the plurality of analysis operation records,
the second record column includes a plurality of subject identifiers associated with the plurality of adoption/non-adoption records, and
the calculation unit calculates the score for each subject based on the first record column and the second record column.

9. A performance management method comprising:
a step of sequentially analyzing a plurality of ultrasound images by using an image analysis model generated through machine learning, thereby sequentially generating a plurality of analysis results;
a step of recording a plurality of analysis operations of the image analysis model and recording adoptions/non-adoptions of the plurality of analysis results to generate a log including a first record column consisting of a plurality of analysis operation records and a second record column consisting of a plurality of adoption/non-adoption records;
a step of calculating a score indicating performance of the image analysis model based on the first record column and the second record column; and
a step of generating reference information to be provided to an examiner in accordance with the score,
wherein the reference information includes at least one of information representing a decrease in the performance of the image analysis model or information for prompting a determination of an adoption/non-adoption of a current analysis result.

10. A non-transitory storage medium storing a program for causing an ultrasound diagnostic apparatus to execute a performance management method, the performance management method including:
a step of sequentially analyzing a plurality of ultrasound images by using an image analysis model generated through machine learning, thereby sequentially generating a plurality of analysis results;
a step of recording a plurality of analysis operations of the image analysis model and recording adoptions/non-adoptions of the plurality of analysis results to generate a log including a first record column consisting of a plurality of analysis operation records and a second record column consisting of a plurality of adoption/non-adoption records;
a step of calculating a score indicating performance of the image analysis model based on the first record column and the second record column; and
a step of generating reference information to be provided to an examiner in accordance with the score,
wherein the reference information includes at least one of information representing a decrease in the performance of the image analysis model or information for prompting a determination of an adoption/non-adoption of a current analysis result.
